(19) United States
(12) Patent Application Publication
(10) Pub. No.: US 2022/0161060 A1
(43) Pub. Date: May 26, 2022

(54) SYSTEM AND METHOD FOR OPTIMIZING RADIOTHERAPHY TREATMENTS

(71) Applicant: CONVERGENT R.N.R LTD., Haifa (IL)

(72) Inventors: Asaf ZUCK, Vardon (IL); Zeev BURSHTEIN, Nes-Ziona (IL); Aharon BAR-DAVID, Nesher (IL); Zeev HAREL, Kfar Saba (IL); Michael KLECKNER, Ramat-Yishai (IL); Shirly BORUKHIN, Atlit (IL)

(21) Appl. No.: 17/601,868

(22) PCT Filed: Apr. 7, 2020
(86) PCT No.: PCT/IL2020/050418

§ 371 Date: Oct. 6, 2021

(30) Foreign Application Priority Data
Apr. 8, 2019 (IL) 265914

(51) Int. Cl. A61N 5/10; A61K 51/12; A61K 51/04

(52) U.S. Cl. CPC A61N 5/1065; A61N 5/1077; A61K 51/0474; A61K 51/1244

(57) ABSTRACT

A radiotherapy treatment system and method used for conducting radiographic X-ray imaging on a target organ during radiographic treatment. The system comprises (a) an x-ray beam source configurable to deliver an X-ray beam to a target organ, (b) optical means for converging and shaping said beam to a cone-shaped X-ray beam of photons which hit the target organ simultaneously, (c) multiple high-Z nanoparticles attachable to the target organ, said high-Z nanoparticles absorbing said X-ray radiation and emitting X-ray fluorescence (XRF) photons, (d) at least one XRF detector for detecting said XRF photons ejecting out of a patient's body, and (e) control means for controlling the radiotherapy treatment procedure.

The x-ray beam is focusable on a section in the target organ where the concentration of said high-Z nanoparticles leading to a desirable emission of said XRF photons, and in case the emission of said XRF photons decreases, the x-ray beam is movable to refocus on the section in the target organ where the emission of said XRF photons is desirable.

### FIELD OF THE INVENTION

The present invention relates to systems and methods of radiotherapy. More specifically, the present invention relates to a system and method for enhancing and optimizing the effects of radiotherapy treatments.

### BACKGROUND OF THE INVENTION

Radiation treatments for tumors, especially cancerous tumors, use high-energy particles or waves, such as x-rays, gamma rays, electron beams, or protons, to destroy or damage cancer cells. Such treatments can have long term negative impacts on a patient due to cell damage in non-tumor regions arising from the high levels of radiation used, thus limiting irradiation to lower doses that are not therapeutic.

Various delivery systems and methods of radiation are available nowadays to reduce damage to healthy tissue surrounding the target cells while keeping the irradiation at high therapeutic doses. However, there is still a need for improved system and method for enhanced radiotherapy treatments that lessen adverse treatment impacts on the patient.

Thus, an aim of the present invention is to provide a system and method for improving and optimizing the effects of radiotherapy treatments while minimizing damage to healthy tissue surrounding the target organ.

### SUMMARY

In accordance with some embodiments of the present invention, there is provided a radiotherapy treatment system for conducting radiographic X-ray imaging on a target organ during radiographic treatment to the target organ. The radiotherapy treatment system comprising:
(i) an x-ray beam source configurable to deliver an X-ray beam to a target organ,
(ii) optical means for converging and shaping said beam to a cone-shaped X-ray beam of photons which hit the target organ simultaneously,
(iii) multiple high-Z nanoparticles/at least one high-Z fiducial marker attachable to said target organ, said high-Z nanoparticles/at least one high-Z fiducial marker absorbing said X-ray radiation and emitting X-ray fluorescence (XRF) photons,
(iv) at least one XRF detector for detecting said XRF photons ejecting out of a patient's body, and
(v) control means for controlling at least one of components (i)-(iv) for controlling said radiotherapy treatment procedure,
   wherein said x-ray beam focusable on a section in said target organ where the concentration of said high-Z nanoparticles leading to a desirable emission of said XRF photons, and
   wherein in case the emission of said XRF photons decreasing, said x-ray beam is movable to refocus on said section in said target organ where the emission of said XRF photons is desirable.

Furthermore, in accordance with some embodiments, the optical means comprising at least one converging lens for converging said X-ray beam to said target organ, Furthermore, in accordance with some embodiments of the present invention, the at least one XRF detector is movable.

Furthermore, in accordance with some embodiments of the present invention, the radiotherapy treatment system further comprising at least one converging lens for converging said XRF photons ejecting out of the patient's body to said at least one XRF detector.

Furthermore, in accordance with some embodiments of the present invention, the at least one XRF detector is selected from a point-sized detector, a one dimensional array detector, and a two-dimensional array detector.

Furthermore, in accordance with some embodiments of the present invention, the point detector is selected from ion chamber type detectors, scintillation detectors and semi-conductor detectors.

Furthermore, in accordance with some embodiments of the present invention, the two-dimensional array detector is a gamma camera.

Furthermore, in accordance with some embodiments of the present invention, the high-Z nanoparticles are selected from metal elements with an atomic number of at least 22.

Furthermore, in accordance with some embodiments of the present invention, the high-Z nanoparticles are selected from titanium (Z=22), vanadium (Z=23), chromium (Z=24), manganese (Z=25), Iron (Z=26), cobalt (Z=27), Nickel (Z=28), copper (Z=29), zinc (Z=30), gallium (Z=31), germanium (Z=32), arsenic (Z=33), selenium (Z=34), bromine (Z=35), rubidium (Z=37), strontium (Z=38), yttrium (Z=39), zirconium (Z=40), niobium (Z=41), molybdenum (Z=42), technetium (Z=43), ruthenium (Z=44), rhodium (Z=45), palladium (Z=46), silver (Z=47), cadmium (Z=48), indium (Z=49), tin (Z=50), antimony (Z=51), tellurium (Z=52), iodine (Z=53), cesium (Z=55), barium (Z=56), lanthanum (Z=57), cerium (Z=58), praseodymium (Z=59), neodymium (Z=60), promethium (Z=61), samarium (Z=62), europium (Z=63), gadolinium (Z=64), terbium (z=65), dysprosium (Z=66) holmium (Z=67), erbium (Z=68), thulium (Z=69) ytterbium (Z=70), lutetium (Z=71), hafnium (Z=72), tantalum (Z=73), tungsten (Z=74), rhenium (Z=75), osmium (Z=76), iridium (Z=77), platinum (Z=78), gold (Z=79), thallium (Z=81), lead (Z=82), bismuth (Z=83), uranium (Z=92).

Furthermore, in accordance with some embodiments of the present invention, the high-Z nanoparticles are preferably selected from Thulium (Z=69) and Erbium (Z=68).

Furthermore, in accordance with some embodiments of the present invention, the high-Z nanoparticles comprise at least one non-metal element.

Furthermore, in accordance with some embodiments of the present invention, the at least one non-metal element is selected from silicone, carbon, halogens, oxygen, and hydrogen.

Furthermore, in accordance with some embodiments of the present invention, the high-Z nanoparticles have a form of nanoscale metal-organic frameworks (nMOFs). Furthermore, in accordance with some embodiments of the present invention, the at least one high-Z nanoparticles comprise Hafnium oxide HfO₂.

Furthermore, in accordance with some embodiments of the present invention, at least two different high-Z nanoparticles are usable, high-Z nanoparticles A and high-Z nanoparticles B, said high-Z nanoparticles A attachable to molecules having affinity to cells of a first type, said high-Z nanoparticles B attachable to molecules having affinity to cells of a second type, wherein the XRF radiation producable by said high-Z nanoparticles A is distinguishable from the XRF radiation producable by said high-Z nanoparticles B.

Furthermore, in accordance with some embodiments of the present invention, in case the XRF radiation producable by said high-Z nanoparticles B decreases and/or the XRF radiation producable by said high-Z nanoparticles A increases during said radiographic treatment procedure, said X-ray beam has to be refocused.

Furthermore, in accordance with some embodiments of the present invention, the cells of a first type being healthy cells and said cells of a second type being non-healthy cells.

Furthermore, in accordance with some embodiments of the present invention, the at least one X-ray detector monitoring in real-time said radiotherapy treatment, thus, providing the distribution of said high-Z nanoparticles in said target organ continuously throughout the radiotherapy treatment.

Furthermore, in accordance with some embodiments of the present invention, the radiotherapy treatment system further comprising a simulation system for simulating said radiographic treatment procedure to maximize the accuracy of the treatment, said simulation system operates independently of the radiotherapy treatment system. Furthermore, in accordance with some embodiments of the present invention, the simulation system comprising an x-ray source, at least one x-ray detector, and multiple high-Z nanoparticles attachable to said target organ.

Furthermore, in accordance with some embodiments of the present invention, the radiotherapy system producing 3D diagnostic images of said target organ, thus, enabling precise treatments.

Furthermore, in accordance with some embodiments of the present invention, the simulation system and said radiotherapy treatment system are usable interchangeably during a treatment to maximize the accuracy of the treatment.
Furthermore, in accordance with some embodiments of the present invention, there is provided a hybrid radiotherapy treatment system for conducting radiographic X-ray imaging on a target organ during radiographic treatment to the target organ and for simulating said radiography treatment to maximize the accuracy of the treatment. The hybrid radiotherapy treatment system comprising:
   (a) an x-ray beam source configurable to deliver an X-ray beam to a target organ,
   (b) optical means for shaping said beam to a cone-shaped X-ray beam of photons which hit the target organ simultaneously,
   (c) multiple high-Z nanoparticles attachable to said target organ, said multiple nanoparticles absorbing said X-ray radiation and emitting X-ray fluorescence (XRF) photons,
   (d) at least one XRF detector for detecting said XRF photons ejecting out of a patient's body,
   (e) an x-ray detector for detecting said x-ray beam passing through said target organ for simulating said radiographic treatment,
   (f) control means for controlling at least one of components (a)-(d), for controlling said simulation and radiotherapy treatment procedures,
      wherein said x-ray beam focusable on a section in said target organ where the concentration of said high-Z nanoparticles leading to a desirable emission of said XRF photons,
      wherein in case the emission of said XRF photons decreasing, said x-ray beam is movable to refocus to said section in said target organ where the emission of said XRF photons is desirable, and
      wherein said hybrid radiotherapy system switching between a simulation mode and a radiotherapy treatment mode without moving a patient from one position to another.

Furthermore, in accordance with some embodiments of the present invention, the optical means comprising at least one lens, said at least one lens comprising an openable aperture, said openable aperture maintained closed for converging said X-ray beam to said target organ during said radiotherapy treatment procedure, said aperture maintained open to allow said beam to pass through said aperture for simulating said radiographic treatment.

Furthermore, in accordance with some embodiments of the present invention, the hybrid radiotherapy system producing 3D diagnostic images of said target organ, thus, enabling precise treatments.

Furthermore, in accordance with some embodiments of the present invention, the at least one XRF detector is movable.

Furthermore, in accordance with some embodiments of the present invention, the hybrid radiotherapy system further comprising at least one converging lens for converging said XRF photons ejecting out of the patient's body to said at least one XRF detector.

Furthermore, in accordance with some embodiments of the present invention, the at least one XRF detector is selected from a point-sized detector, a one dimensional array detector, and a two-dimensional array detector.

Furthermore, in accordance with some embodiments of the present invention, the point-sized detector is selected from ion chamber type detectors, scintillation detectors and semi-conductor detectors.

Furthermore, in accordance with some embodiments of the present invention, the two-dimensional array detector is a gamma camera.

Furthermore, in accordance with some embodiments of the present invention, the high-Z nanoparticles are selected from metal elements with an atomic number of at least 22.

Furthermore, in accordance with some embodiments of the present invention, the high-Z nanoparticles are selected from titanium (Z=22), vanadium (Z=23), chromium (Z=24), manganese (Z=25), Iron (Z=26), cobalt (Z=27), Nickel (Z=28), copper (Z=29), zinc (Z=30), gallium (Z=31), germanium (Z=32), arsenic (Z=33), selenium (Z=34), bromine (Z=35), rubidium (Z=37), strontium (Z=38), yttrium (Z=39), zirconium (Z=40), niobium (Z=41), molybdenum (Z=42), technetium (Z=43), ruthenium (Z=44), rhodium (Z=45), palladium (Z=46), silver (Z=47), cadmium (Z=48), indium (Z=49), tin (Z=50), antimony (Z=51), tellurium (Z=52), iodine (Z=53), cesium (Z=55), barium (Z=56), lanthanum (Z=57), cerium (Z=58), praseodymium (Z=59), neodymium (Z=60), promethium (Z=61), samarium (Z=62), europium (Z=63), gadolinium (Z=64), terbium (z=65), dysprosium (Z=66) holmium (Z=67), erbium (Z=68), thulium (Z=69) ytterbium (Z=70), lutetium (Z=71), hafnium (Z=72), tantalum (Z=73), tungsten (Z=74), rhenium (Z=75), osmium (Z=76), iridium (Z=77), platinum (Z=78), gold (Z=79), thallium (Z=81), lead (Z=82), bismuth (Z=83), uranium (Z=92).

Furthermore, in accordance with some embodiments of the present invention, the high-Z nanoparticles are preferably selected from Thulium (Z=69) and Erbium (Z=68).

Furthermore, in accordance with some embodiments of the present invention, the high-Z nanoparticles comprise at least one non-metal element.

Furthermore, in accordance with some embodiments of the present invention, the at least one non-metal element is selected from silicone, halogens, oxygen, and hydrogen.

Furthermore, in accordance with some embodiments of the present invention, the at least one high-Z nanoparticles comprise Hafnium oxide HfO₂.

Furthermore, in accordance with some embodiments of the present invention, at least two types, a first type and a second type, of said high-Z nanoparticles are being used, said high-Z nanoparticles of said first type being attached to molecules having affinity to one kind (e.g. healthy cells), said high-Z nanoparticles of said second type being attached to molecules having affinity to other kind (e.g. non-healthy) of cells, so that, the XRF radiation produced by said high-Z nanoparticles of said first type is distinguishable from the XRF radiation produced by said high-Z nanoparticles of said second type.

Furthermore, in accordance with some embodiments of the present invention, the at least one X-ray detector monitoring in real-time said radiotherapy treatment procedure, thus, providing the distribution of said high-Z nanoparticles in said target organ continuously throughout the radiotherapy treatment.

Furthermore, in accordance with some embodiments of the present invention, there is also provided a radiotherapy treatment method for conducting a radiographic X-ray imaging on a target organ in real time during a radiation treatment procedure. The method comprises:
providing the above described radiotherapy treatment system;
administrating at least one high-Z metal nanoparticle to a target organ in a patient's body;
delivering radiation via an X-ray beam to the target organ;
emitting XRF photons from the high-Z nanoparticles;
detecting said XRF photons;
directing said X-ray beam's focal point; and
moving and refocusing said x-ray beam when detecting a decrease in the emission of said XRF photons.

Furthermore, in accordance with some embodiments of the present invention, moving and refocusing said x-ray beam to a section in the target organ where the concentration of the high-Z metal nanoparticles is desirable.

Furthermore, in accordance with some embodiments of the present invention, the method further comprises simulating said radiographic a treatment procedure for obtaining a distribution of said high-Z nanoparticles to maximize the accuracy of the treatment.

### BRIEF DESCRIPTION OF THE FIGURES

FIG. 1 illustrates a radiotherapy treatment system for conducting a radiographic X-ray imaging on a target organ during a radiation treatment in accordance with some embodiments of the present invention.

FIG. 2 illustrates a hybrid system having both simulation and treatment capabilities in accordance with some embodiments of the present invention.

FIG. 3 illustrates a radiotherapy simulation system for simulating radiographic treatments to enable precise radiotherapy treatments in accordance with some embodiments of the present invention.

FIG. 4 illustrates a flowchart of a method for conducting XRF imaging on a target organ during a radiation treatment in accordance with some embodiments of the present invention.

### DETAILED DESCRIPTION OF THE FIGURES

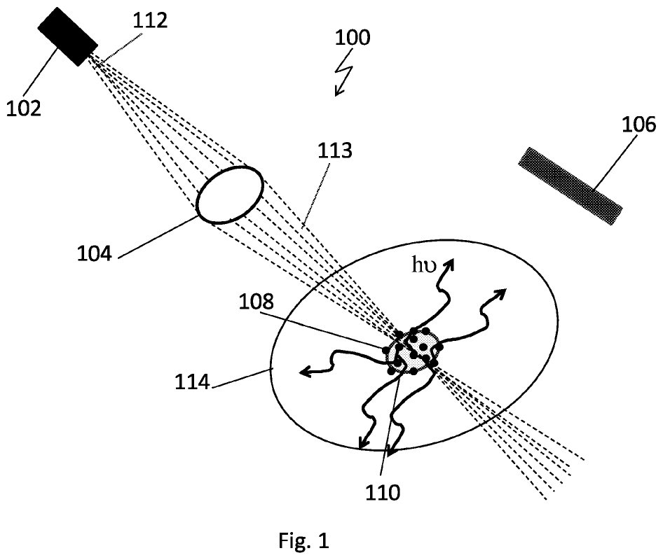

FIG. 1 illustrates a radiotherapy treatment system 100 for conducting a radiographic X-ray imaging on a target organ during a radiation treatment in accordance with some embodiments of the present invention.

Radiotherapy treatment system 100 is used for treating target organs which can be a cancerous tumor or a non-cancerous lesion or other organ while at the same time monitoring in real-time the radiotherapy process and verifying that the radiation “snap” is directed towards a desired location.

Radiotherapy treatment system 100 of the present invention comprises an X-ray beam source 102, a converging lens 104, at least one X-ray detector 106, and either multiple high-Z nanoparticles 108 or at least one high-Z fiducial marker 109.

Radiotherapy treatment system 100 may also include an additional converging lens for converging the emitted XRF photons towards X-ray detector 106.

In accordance with some embodiments of the present invention, high-Z nanoparticles 108 or at least one high-Z fiducial marker 109 are inside and/or on the outside surface of target organ 110.

In accordance with some embodiments of the present invention, all components of radiotherapy treatment system 100 are spatially movable to various target organs in the body (e.g., from head to toe). In accordance with some embodiments of the present invention, at least one X-ray detector 106 may be spatially movable in order to monitor the radiation therapy from various positions around the patient. Alternatively, multiple units of the at least one X-ray detector 106 may be stationary at various positions in the vicinity of the patient for monitoring the radiation therapy from various positions around the patient.

The at least one X-ray detector 106 may be selected from a point-sized detector such as an ion chamber type detector, a scintillating detector, or a semi-conductor detector. Alternatively, the at least one X-ray detector 106 may be selected from a line array detector, and a two-dimensional array detector such as a gamma camera.

Unlike point detectors which have to be moved in space to get mapping, line array detectors and two-dimensional detectors give instantaneous spatial data of the XRF emitted from the body. Such data is highly essential for directing and monitoring the radiation therapy.

In accordance with some embodiments of the present invention, the X-ray beam source 102 emits a diverging X-ray beam 112 which is targeted towards a first converging lens 104 that converges the beam towards a target organ 110 within a patient's body 114.

Prior to the radiation treatment, target organ 110 is administrated with either at least one high-Z fiducial marker 109 or high-Z nanoparticles 108 selected from metal elements with an atomic number of at least 22, including but not limited to titanium (Z=22), vanadium (Z=23), chromium (Z=24), manganese (Z=25), Iron (Z=26), cobalt (Z=27), Nickel (Z=28), copper (Z=29), zinc (Z=30), gallium (Z=31), germanium (Z=32), arsenic (Z=33), selenium (Z=34), bromine (Z=35), rubidium (Z=37), strontium (Z=38), yttrium (Z=39), zirconium (Z=40), niobium (Z=41), molybdenum (Z=42), technetium (Z=43), ruthenium (Z=44), rhodium (Z=45), palladium (Z=46), silver (Z=47), cadmium (Z=48), indium (Z=49), tin (Z=50), antimony (Z=51), tellurium (Z=52), iodine (Z=53), cesium (Z=55), barium (Z=56), lanthanum (Z=57), cerium (Z=58), praseodymium (Z=59), neodymium (Z=60), promethium (Z=61), samarium (Z=62), europium (Z=63), gadolinium (Z=64), terbium (z=65), dysprosium (Z=66) holmium (Z=67), erbium (Z=68), thulium (Z=69) ytterbium (Z=70), lutetium (Z=71), hafnium (Z=72), tantalum (Z=73), tungsten (Z=74), rhenium (Z=75), osmium (Z=76), iridium (Z=77), platinum (Z=78), gold (Z=79), thallium (Z=81), lead (Z=82), bismuth (Z=83), uranium (Z=92).

In accordance with some embodiments of the present invention, the use of high-Z nanoparticles of either non-toxic Thulium (Z=69) or Erbium (Z=68) which has low toxicity is preferred in some cases due to a good match between the K-edge absorption resonance energy of these materials, with the emission characteristic of Tungsten. The K-edge of Tellurium—Tm is 59.3896 keV, the K-edge of Erbium—Er is 57.4855 keV, and the Ka emission of Tungsten, which is the most commonly used anode material in X-ray tubes for medical applications, is 59.3 keV.

It should be noted that non-metal components such as silicone, carbon. halogens (such as iodine), oxygen, and even hydrogen may be present in the compound, an example is the Hafnium oxide HfO₂.

It should be further noted that the high-Z nanoparticles may appear in the form of nanoscale metal-organic frameworks (nMOFs).

In accordance with some embodiments of the present invention, the distribution of such nanoparticles is obtained during the radiation treatment via the at least one X-ray detector 106 detecting the emitted XRF photons from said high-Z nanoparticles 108. The high-Z nanoparticles 108/high-Z fiducial marker 109 absorb the X-ray radiation and emit X-ray fluorescence (XRF) photons where a fraction of sufficiently high energy photons emerges out of the patient's body and detected by X-ray detector 106. The XRF emission intensity depends on the energy and intensity of the radiation and the concentration of the high-Z nanoparticles 108. In the simplest case, it is assumed that the XRF emission is maximal when the converged radiation focal point hits the highest concentration of high-Z nanoparticles 108.

In accordance with some embodiments of the present invention, the at least one X-ray detector 106 provides information about the actual distribution of nanoparticles, i.e., monitors a real-time radiotherapy process to provide the distribution of nanoparticles in the area of interest continuously throughout the radiotherapy treatment. Such information is highly essential for verifying that the radiation “snap” is indeed directed towards the exact location (spatial coordination), i.e., towards the target organ having a high density of high-Z nanoparticles 108.

When the XRF emission decreases, it is required to check whether the beam's focal point is in the correct place. If the beam is out of focus, it has to be directed to the section where the XRF radiation received is at a desirable value.

In accordance with some embodiments of the present invention, the use of a converging X-ray beam 113 and high-Z nanoparticles 108 enables the target organ 110 to receive a large dosage while the distal and preceding organs receive minimal dosage. Minimizing the dosage to healthy tissues reduces the amount of side effects that the patient will suffer from. Therefore, the use of a converging beam 113 and high-Z nanoparticles 108 reduces long term as well as short term side effects. In addition, the increased dose supplied to the target organ 110 increases the efficiency of the therapy and might even shorten the therapy course making it more tolerable to patients.

In accordance with some embodiments of the present invention, target organ 110 may be administrated with high-Z nanoparticles 108 of various elements. Prior to being administrated, high-Z nanoparticles 108A of certain element(s) may be attached to molecules having an affinity to cells of a first type while high-Z nanoparticles 108B of other element(s) may be attached to molecules having an affinity to cells of a second type. Since high-Z nanoparticles 108 of different elements produce different XRF radiations (different energies), such configuration enables determining the particle distribution in cells of both types and focusing the X-ray beam 112 on the desired cells.

For instance, high-Z nanoparticles 108A of certain element(s) may be attached to molecules having an affinity to healthy cells while high-Z nanoparticles 108B of other element(s) may be attached to molecules having an affinity to non-healthy cells. As long as the beam is focused, the XRF emitted from the high-Z nanoparticles 108B may be dominant. However, if during the treatment procedure the XRF emitted from the high-Z nanoparticles 108B is no longer dominant, i.e., if the XRF emitted from high-Z nanoparticles 108B decreases and/or the XRF emitted from high-Z nanoparticles 108A increases, it is most likely due to a beam shift from the initial location, and thus, the beam has to be refocused to its original location. In accordance with some embodiments of the present invention, radiotherapy treatment system 100 may include a simulator for providing a radiographic reflection of the distribution of high-Z nanoparticles 108 in target organ 110, thus, for verifying treatment plans with high accuracy.

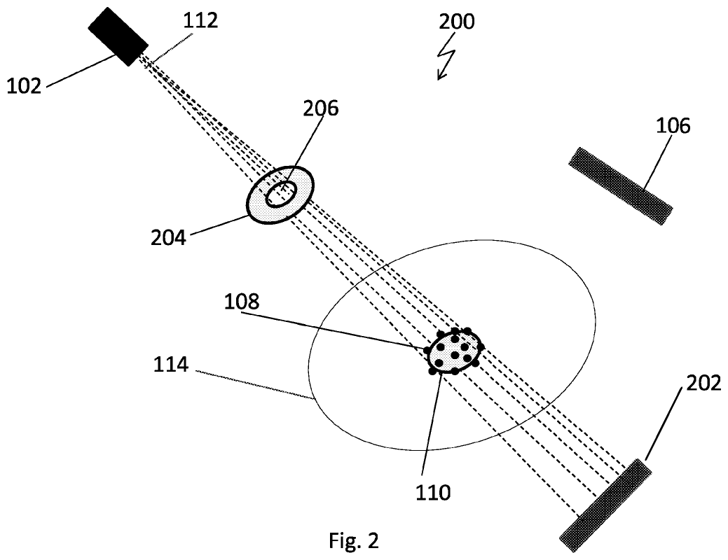

FIG. 2 illustrates a hybrid system 200 having both simulation and treatment capabilities in accordance with some embodiments of the present invention. Hybrid system 200 is basically the radiotherapy treatment system 100 of FIG. 1 modified to implement simulation capabilities for simulating of target organ 110, and thus, for enabling precise treatment procedures.

Hybrid system 200 can quickly switch between a radiographic simulation mode and a radiotherapy X-ray treatment mode without moving the patient from the actual treatment position, i.e., patients can be imaged in the true treatment position.

Hybrid system 200 comprises a converging X-ray beam source 102, at least one X-ray detector 106, target organ 110 to be detected by X-ray detector 202, high-Z nanoparticles 108 or at least one high-Z fiducial marker 109 attached inside and/or on the outside surface of the target organ 110, lens 204, and a second detector 202 for detecting beam 112 traveling through lens 204 during simulations runs.

In accordance with some embodiments of the present invention, lens 204 comprises an aperture 206 through which X-ray Beam 112 passes towards target organ 110 and detected via X-ray detector 202 during simulation runs.

In accordance with some embodiments of the present invention, simulations are run prior to a radiation treatment for focusing the X-ray beam 112 without moving the patient from the actual treatment position.

In order to avoid damages to target organ 110 or any other organs in the vicinity of the target organ 110, X-ray beam 112 passes through aperture 206 of lens 204, and thus, does not converge into the organ during the simulation process.

In accordance with some embodiments of the present invention, hybrid system 200 produces 3D diagnostic images of target organ 110, and thus, enables precise treatments. At the end of the simulation process, System 200 is being changed from radiographic simulating mode for beam focusing to a radiotherapy X-ray treatment mode without moving the patient from the actual treatment position.

In accordance with some embodiments of the present invention, hybrid system 200 can quickly change from a radiographic simulation mode to a radiotherapy X-ray treatment mode and vice versa without moving the patient from the actual treatment position, i.e., patients can be imaged in the true treatment position.

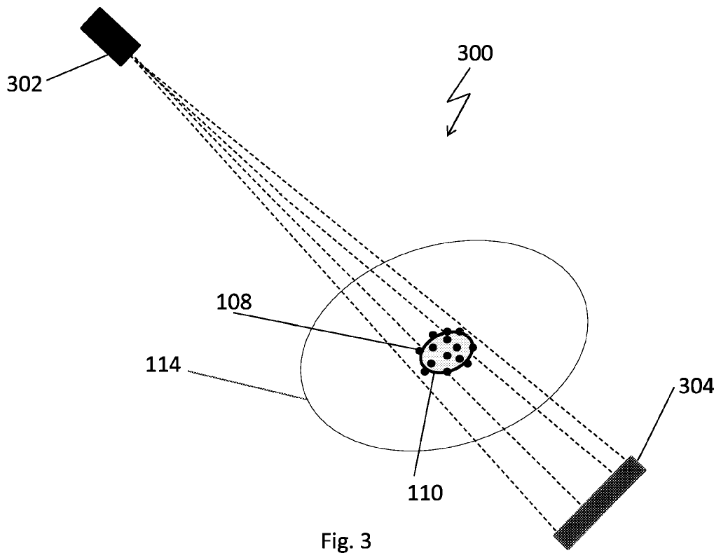

FIG. 3 illustrates a radiotherapy simulation system 300 for simulating radiographic treatments in accordance with some embodiments of the present invention.

In this case, radiotherapy simulation system 300 is a separate, stand-alone system which operates independently of radiotherapy treatment system 100.

Radiotherapy simulation system 300 comprising an X-ray beam source 302 which is not that of the therapeutic beam but rather an independent beam source 302, at least one X-ray detector 304, and high-Z nanoparticles 108/at least one high-Z fiducial marker 109 attached inside and on the outside surface of the target organ 110.

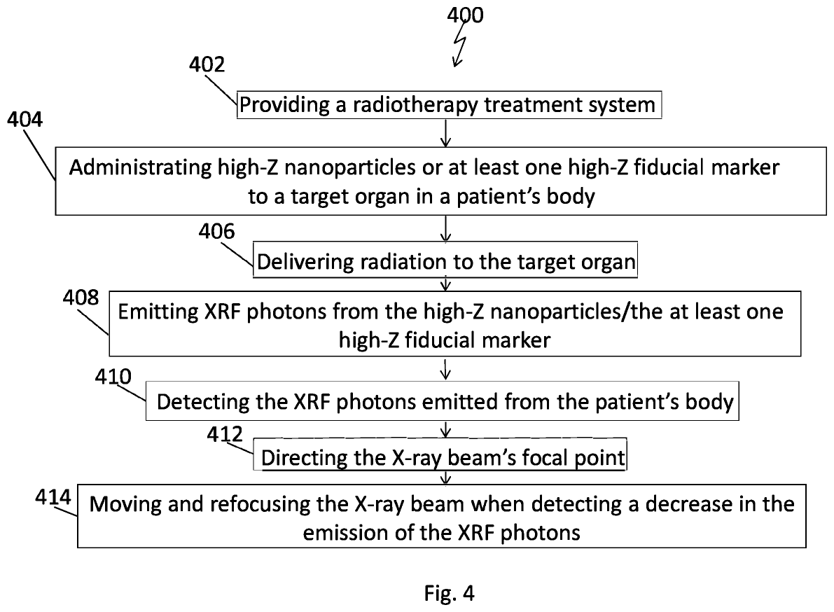

In accordance with some embodiments of the present invention, radiotherapy treatment system 100 of FIG. 1 and radiotherapy simulation system 300 may be used interchangeably during a treatment in order to maximize the accuracy of the treatment. FIG. 4 illustrates a flowchart of a method 400 for conducting XRF imaging on a target organ 110 during a radiation treatment in accordance with some embodiments of the present invention.

The method 400 begins with step 402 in which a radiotherapy treatment system is provided.

In administering step 404, an effective amount of high-Z nano-particles 108 or at least one high-Z fiducial marker 109 is localized in the target organ 110 via (a) injection into the blood stream with or without affinity agents to aid in localization, (b) direct injection into the target organ 110 or any other administering step that is capable of localizing an effective amount of the high-Z nanoparticles 108 in the target organ 110. X-ray radiation is delivered to the target organ 110 in step 406. As radiation is applied to the target organ 110 and absorbed by the high-Z nano-particles 108, a portion of the radiation produces energized electrons such as photoelectrons, Compton electrons, or the like, is ejected from the NP and deliver their energy to the target organ.

Another process is X ray fluorescence (XRF) where photons having specific energies are emitted from the high-Z nano-particles 108 or the at least one high-Z fiducial marker 109 in step 408. The emitted energy is delivered via energetic electrons and photons to the surrounding environment (the tumor), thereby increasing the efficiency of therapeutic dose delivering to the tumor—the photons and electrons are absorbed by the tissues of the target organ resulting in cell death or a reduction or elimination of a tumor in a reducing cancer cells.

A fraction of sufficiently high energy XRF photons emerges out of the patient's body 114 and collected by at least one detector 106 located at different positions in the patient vicinity in step 410 to determine the exact distribution of the nanoparticles 108.

The focal point of the X-ray beam 112 is directed to the target area in step 412 as follows:
X-ray radiation is delivered to the target organ 110,
the primary (therapeutic) X ray beam 112 causes an emission of XRF photons via interaction of the high-Z nanoparticles 108,
   photons ejected from the patient's body 114 are detected continuously throughout the treatment, the X-ray beam 112 is focused on the target organ 110 and preferably on a section in the target organ 110 where the concentration of the high-Z nanoparticles 108 lead to a desirable emission of the XRF photons.

Thus, in accordance with some embodiments of the present invention, the XRF photons ejected from the human's body are detected continuously throughout the radiotherapy treatment, and the X-ray beam is automatically refocused whenever a decrease in the emission of the XRF photons is detected.

When the reading of detector 106 is less than a pre-defined value, it is assumed that X-ray beam 112 either hits a region within the target organ 110 where the concentration of high-Z nanoparticles 108 is undesirable or there is greater absorption in the patient's body 114. This means that the focus position is not optimal and therefore the direction of X-ray beam 112 is corrected in step 414.

In accordance with some embodiments of the present invention, returning the X-ray beam 112 to its original location involves with scanning the target organ 110 to locate the point where the XRF emission is desirable and returning the therapeutic radiation to the new coordinates.

In accordance with some embodiments of the present invention, method 400 may further comprise simulation runs to obtain a distribution of the high-Z nanoparticles 108 in the target organ 110 prior to delivering therapeutic radiation to the target organ. Alternatively, the skin may be marked via markers or tattoos to help directing the beam 112 to a desired location.

In accordance with some embodiments of the present invention, the desired focal point of the beam 112, is determined via straight-forward triangulation in step 410. Triangulation is a process of determining the location of a point by forming triangles to it from known points. Basically, the configuration consists of either a single movable sensor or multiple sensors for observing the item, i.e., the XRF emitted from said high-Z nanoparticles. One of the sensors is typically a point detector, or one-dimensional array detector, or two-dimensional array detector, and the other sensor may be either a point detector or one-dimensional array detector, or two-dimensional array detector or a new location of the detector and the third is the XRF emitter. The projection centers of the sensors and a considered point on the NP loaded target organ's surface define a triangle. Within this triangle, the distance between the sensors is the base b and must be known. By determining the angles between the projection rays of the sensors and the basis, the intersection point, and thus the 3D coordinate, is calculated from the triangular relations.

1. A radiotherapy treatment system for conducting radiographic X-ray imaging on a target organ during radiographic treatment to the target organ comprising:
(i) an x-ray beam source configurable to deliver an X-ray beam to a target organ,
(ii) optical means for converging and shaping said beam to a cone-shaped X-ray beam of photons which hit the target organ simultaneously,
(iii) multiple high-Z nanoparticles/at least one high-Z fiducial marker attachable to said target organ, said high-Z nanoparticles/at least one high-Z fiducial marker absorbing said X-ray radiation and emitting X-ray fluorescence (XRF) photons,
(iv) at least one XRF detector for detecting said XRF photons ejecting out of a patient's body, and
(v) control means for controlling at least one of components (i)-(iv) for controlling said radiotherapy treatment procedure,
wherein said x-ray beam focusable on a section in said target organ where the concentration of said high-Z nanoparticles leading to a desirable emission of said XRF photons, and
wherein in case the emission of said XRF photons decreasing, said x-ray beam is movable to refocus on said section in said target organ where the emission of said XRF photons is desirable.

2. The radiotherapy treatment system of claim 1, wherein said optical means comprising at least one converging lens for converging said X-ray beam to said target organ,

3. The radiotherapy treatment system of claim 1, wherein said at least one XRF detector is movable.

4. The radiotherapy treatment system of claim 1, further comprising at least one converging lens for converging said XRF photons ejecting out of the patient's body to said at least one XRF detector.

5. The radiotherapy treatment system of any one of claims 1-4, wherein said at least one XRF detector selected from a point-sized detector, a one dimensional array detector, and a two-dimensional array detector.

6. The radiotherapy treatment system of claim 5, wherein said point detector is selected from ion chamber type detectors, scintillation detectors and semi-conductor detectors.

7. The radiotherapy treatment system of claim 6, wherein said two-dimensional array detector is a gamma camera.

8. The radiotherapy treatment system of claim 1, wherein said high-Z nanoparticles are selected from metal elements with an atomic number of at least 22.

9. The radiotherapy treatment system of claim 8, wherein said high-Z nanoparticles are selected from titanium (Z=22), vanadium (Z=23), chromium (Z=24), manganese (Z=25), Iron (Z=26), cobalt (Z=27), Nickel (Z=28), copper (Z=29), zinc (Z=30), gallium (Z=31), germanium (Z=32), arsenic (Z=33), selenium (Z=34), bromine (Z=35), rubidium (Z=37), strontium (Z=38), yttrium (Z=39), zirconium (Z=40), niobium (Z=41), molybdenum (Z=42), technetium (Z=43), ruthenium (Z=44), rhodium (Z=45), palladium (Z=46), silver (Z=47), cadmium (Z=48), indium (Z=49), tin (Z=50), antimony (Z=51), tellurium (Z=52), iodine (Z=53), cesium (Z=55), barium (Z=56), lanthanum (Z=57), cerium (Z=58), praseodymium (Z=59), neodymium (Z=60), promethium (Z=61), samarium (Z=62), europium (Z=63), gadolinium (Z=64), terbium (z=65), dysprosium (Z=66) holmium (Z=67), erbium (Z=68), thulium (Z=69) ytterbium (Z=70), lutetium (Z=71), hafnium (Z=72), tantalum (Z=73), tungsten (Z=74), rhenium (Z=75), osmium (Z=76), iridium (Z=77), platinum (Z=78), gold (Z=79), thallium (Z=81), lead (Z=82), bismuth (Z=83), uranium (Z=92).

10. The radiotherapy treatment system of claim 9, wherein said high-Z nanoparticles are preferably selected from Thulium (Z=69) and Erbium (Z=68).

11. The radiotherapy treatment system of any one of claims 1, 8-10, wherein said high-Z nanoparticles comprising at least one non-metal element.

12. The radiotherapy treatment system of claim 11, wherein said at least one non-metal element is selected from silicone, carbon, halogens, oxygen, and hydrogen.

13. The radiotherapy treatment system of any one of claims 1, and 8-12, wherein said high-Z nanoparticles having a form of nanoscale metal-organic frameworks (nMOFs).

14. The radiotherapy treatment system of any one of claims 1, 8-10, wherein said at least one high-Z nanoparticles comprising Hafnium oxide HfO₂.

15. The radiotherapy treatment system of any one of claims 1, 8-14, wherein at least two different high-Z nanoparticles are usable, high-Z nanoparticles A and high-Z nanoparticles B, said high-Z nanoparticles A attachable to molecules having affinity to cells of a first type, said high-Z nanoparticles B attachable to molecules having affinity to cells of a second type, wherein the XRF radiation producable by said high-Z nanoparticles A is distinguishable from the XRF radiation producable by said high-Z nanoparticles B.

16. The radiotherapy treatment system of claim 15, wherein in case the XRF radiation producable by said high-Z nanoparticles B decreases and/or the XRF radiation producable by said high-Z nanoparticles A increases during said radiographic treatment procedure, said X-ray beam has to be refocused.

17. The radiotherapy treatment system of any one of claims 15-16, wherein said cells of a first type being healthy cells and said cells of a second type being non-healthy cells.

18. The radiotherapy treatment system of any one of claims 1, 3-7, wherein said at least one X-ray detector monitoring in real-time said radiotherapy treatment, thus, providing the distribution of said high-Z nanoparticles in said target organ continuously throughout the radiotherapy treatment.

19. The radiotherapy treatment system of any one of claims 1-18, further comprising a simulation system for simulating said radiographic treatment procedure to maximize the accuracy of the treatment, said simulation system operates independently of the radiotherapy treatment system.

20. The radiotherapy treatment system of claim 19, wherein said simulation system comprising an x-ray source, at least one x-ray detector, and multiple high-Z nanoparticles attachable to said target organ.

21. The radiotherapy treatment system of any one of claims 18-20, wherein said radiotherapy system producing 3D diagnostic images of said target organ, thus, enabling precise treatments.

22. The radiotherapy treatment system of any one of claims 19-21, wherein said simulation system and said radiotherapy treatment system are usable interchangeably during a treatment to maximize the accuracy of the treatment.

23. A hybrid radiotherapy treatment system for conducting radiographic X-ray imaging on a target organ during radiographic treatment to the target organ and for simulating said radiography treatment to maximize the accuracy of the treatment, said hybrid radiotherapy treatment system comprising:
(a) an x-ray beam source configurable to deliver an X-ray beam to a target organ,
(b) optical means for shaping said beam to a cone-shaped X-ray beam of photons which hit the target organ simultaneously,
(c) multiple high-Z nanoparticles/at least one high-Z fiducial marker attachable to said target organ, said multiple nanoparticles/at least one high-Z fiducial marker absorbing said X-ray radiation and emitting X-ray fluorescence (XRF) photons,
(d) at least one XRF detector for detecting said XRF photons ejecting out of a patient's body,
(e) an x-ray detector for detecting said x-ray beam passing through said target organ for simulating said radiographic treatment,
(f) control means for controlling at least one of components (a)-(d), for controlling said simulation and radiotherapy treatment procedures,
wherein said x-ray beam focusable on a section in said target organ where the concentration of said high-Z nanoparticles leading to a desirable emission of said XRF photons,
wherein in case the emission of said XRF photons decreasing, said x-ray beam is movable to refocus to said section in said target organ where the emission of said XRF photons is desirable, and
wherein said hybrid radiotherapy system switching between a simulation mode and a radiotherapy treatment mode without moving a patient from one position to another.

24. The hybrid radiotherapy system of claim 23, wherein said optical means comprising at least one lens, said at least one lens comprising an openable aperture, said openable aperture maintained closed for converging said X-ray beam to said target organ during said radiotherapy treatment procedure, said aperture maintained open to allow said beam to pass through said aperture for simulating said radiographic treatment.

25. The hybrid radiotherapy system of claim 23, wherein said hybrid radiotherapy system producing 3D diagnostic images of said target organ, thus, enabling precise treatments.

26. The hybrid radiotherapy system of claim 23, wherein said at least one XRF detector is movable.

27. The hybrid radiotherapy system of claim 23, further comprising at least one converging lens for converging said XRF photons ejecting out of the patient's body to said at least one XRF detector.

28. The hybrid radiotherapy system of any one of claims 23-26, wherein said at least one XRF detector selected from a point-sized detector, a one dimensional array detector, and a two-dimensional array detector.

29. The hybrid radiotherapy system of claim 27, wherein said point-sized detector is selected from ion chamber type detectors, scintillation detectors and semi-conductor detectors.

30. The hybrid radiotherapy system of claim 27, wherein said two-dimensional array detector is a gamma camera.

31. The hybrid radiotherapy system of claim 23, wherein said high-Z nanoparticles are selected from metal elements with an atomic number of at least 22.

32. The hybrid radiotherapy system of claim 31, wherein said high-Z nanoparticles are selected from titanium (Z=22), vanadium (Z=23), chromium (Z=24), manganese (Z=25), Iron (Z=26), cobalt (Z=27), Nickel (Z=28), copper (Z=29), zinc (Z=30), gallium (Z=31), germanium (Z=32), arsenic (Z=33), selenium (Z=34), bromine (Z=35), rubidium (Z=37), strontium (Z=38), yttrium (Z=39), zirconium (Z=40), niobium (Z=41), molybdenum (Z=42), technetium (Z=43), ruthenium (Z=44), rhodium (Z=45), palladium (Z=46), silver (Z=47), cadmium (Z=48), indium (Z=49), tin (Z=50), antimony (Z=51), tellurium (Z=52), iodine (Z=53), cesium (Z=55), barium (Z=56), lanthanum (Z=57), cerium (Z=58), praseodymium (Z=59), neodymium (Z=60), promethium (Z=61), samarium (Z=62), europium (Z=63), gadolinium (Z=64), terbium (z=65), dysprosium (Z=66) holmium (Z=67), erbium (Z=68), thulium (Z=69) ytterbium (Z=70), lutetium (Z=71), hafnium (Z=72), tantalum (Z=73), tungsten (Z=74), rhenium (Z=75), osmium (Z=76), iridium (Z=77), platinum (Z=78), gold (Z=79), thallium (Z=81), lead (Z=82), bismuth (Z=83), uranium (Z=92).

33. The radiotherapy treatment system of claim 32, wherein said high-Z nanoparticles are preferably selected from Thulium (Z=69) and Erbium (Z=68).

34. The hybrid radiotherapy system of any one of claims 23, 32, & 33, wherein said high-Z nanoparticles comprising at least one non-metal element.

35. The hybrid radiotherapy system of claim 34, wherein said at least one non-metal element is selected from silicone, halogens, oxygen, and hydrogen.

36. The hybrid radiotherapy treatment system of any one of claims 23, 32-33, wherein said at least one high-Z nanoparticles comprising Hafnium oxide HfO₂.

37. The hybrid radiotherapy treatment system of any one of claims 23 and 32-36, wherein at least two types, a first type and a second type, of said high-Z nanoparticles being used, said high-Z nanoparticles of said first type being attached to molecules having affinity to one kind (e.g. healthy cells), said high-Z nanoparticles of said second type being attached to molecules having affinity to other kind (e.g. non-healthy) of cells, so that, the XRF radiation produced by said high-Z nanoparticles of said first type is distinguishable from the XRF radiation produced by said high-Z nanoparticles of said second type.

38. The hybrid radiotherapy system of any one of claims 22, 25-29, wherein said at least one X-ray detector monitoring in real-time said radiotherapy treatment procedure, thus, providing the distribution of said high-Z nanoparticles in said target organ continuously throughout the radiotherapy treatment.

39. A radiotherapy treatment method for conducting a radiographic X-ray imaging on a target organ in real time during a radiation treatment procedure comprising:
providing the radiotherapy treatment system of claims 1-13;
administrating at least one high-Z metal nanoparticle or at least one high-Z fiducial marker to a target organ in a patient's body;
delivering radiation via an X-ray beam to the target organ;
emitting XRF photons from the high-Z nanoparticles/the at least one high-Z fiducial marker;
detecting said XRF photons;
directing said X-ray beam's focal point; and
moving and refocusing said x-ray beam when detecting a decrease in the emission of said XRF photons.

40. The radiotherapy treatment method of claim 39, wherein moving and refocusing said x-ray beam to a section in the target organ where the concentration of the high-Z metal nanoparticles is desirable.

41. The method of any one of claims 39 and 40 further comprising simulating said radiographic treatment procedure for obtaining a distribution of said high-Z nanoparticles to maximize the accuracy of the treatment.
